(19) United States
(12) Patent Application Publication
(10) Pub. No.: US 2005/0112652 A1
(43) Pub. Date: May 26, 2005

(54) Device and method for detecting biochemical reactions and/or bindings

(75) Inventors: Stephan Dertinger, Munchen (DE); Michaela Fritz, Munchen (DE); Karin Fuchs, Bach (DE); Thomas Haneder, Dachau (DE); Hans-Christian Hanke, Munchen (DE); Alfred Martin, Munchen (DE); Reinhard Marz, Munchen (DE)

Correspondence Address: DARBY & DARBY P.C., P. O. BOX 5257, NEW YORK, NY, 10150-5257, US

(73) Assignee: Infineon Technologies AG, Munich (DE)

(21) Appl. No.: 10/968,847

(22) Filed: Oct. 19, 2004

Related U.S. Application Data
(63) Continuation of application No. PCT/EP03/03997, filed on Apr. 16, 2003.

(30) Foreign Application Priority Data
Apr. 19, 2002 (DE) 102 17 568.3

(51) Int. Cl. C12Q 1/68

(52) U.S. Cl. 435/6

(57) ABSTRACT

Device having a two-dimensionally formed support material which has, spread across at least one surface area, a plurality of pores which stretch throughout from one surface of the support material to the opposite surface, wherein the pores are bound in each case by a pore boundary area of pore walls formed in the support material along particular longitudinal axes of the pores, and at least part of the pore walls have at least in some sections a layered structure containing a first layer forming the pore boundary area and a second layer adjacent to the first layer and spaced apart from the pore boundary area, and wherein the refractive index n_{waveguide} of the first layer is greater than the refractive index n₂ of the second layer.

### CROSS-REFERENCE TO RELATED APPLICATION

This application is a continuation of International Patent Application Serial No. PCT/EP03/03997, filed Apr. 16, 2003, which published in German on Oct. 30, 2003 as WO 03/089931, and is incorporated herein by reference in its entirety.

### FIELD OF THE INVENTION

The present invention relates to a device having a two-dimensionally formed support material which has, spread across at least one surface area, a multiplicity of pores which stretch throughout from one surface of the support material to the opposite surface, to the use of the device as a basis for a sample support in methods of detecting biochemical reactions and/or bindings, to methods of detecting chemical or biochemical reactions and/or bindings, and to a method of preparing the device.

### BACKGROUND OF THE INVENTION

The devices of the invention are suitable as bases for “biochip base modules” in methods of detecting biochemical (binding) reactions and also, to this end in particular, for studying enzymic reactions, nucleic acid hybridizations, protein-protein interactions and other binding reactions in the field of genome, proteome or drug research in biology and medicine.

Biochips which are used to obtain rapidly knowledge about organisms and tissues are nowadays increasingly employed in molecular biology. The detection of (bio) chemical reactions, i.e. detection of biologically relevant molecules in defined test material, is extraordinarily important for the biosciences and medical diagnostics. In this context, the development of “biochips” is being constantly pursued. Such biochips are usually miniaturized hybrid functional elements with biological and technical components, in particular biomolecules which are immobilized on a surface of a biochip base module and which are used as specific interaction partners. These functional elements are frequently structured in the form of rows and columns and are then called “microarrays”. Since thousands of biological or biochemical functional elements may be arranged on one chip, they are generally fabricated using microtechnical methods.

Particularly suitable biological and biochemical functional elements are DNA, RNA, PNA (in nucleic acids and their chemical derivatives, for example, single strands such as oligonucleotides, triplex structures or combinations thereof may be present), saccharides, peptides, proteins (e.g. antibodies, antigens, receptors), derivatives from combinatorial chemistry (e.g. organic molecules), cell components (e.g. organelles), individual cells, multicellular organisms and cell assemblages.

“Microarrays” are the most widespread biochip variants. They are small wafers (“chips”) of glass, gold, plastic or silicon, for example. In order to detect corresponding biological or biochemical (binding) reactions, for example, small amounts of solubilized different capture molecules, for example a known nucleic acid sequence, are fixed on the surface of the biochip base module in the form of very small droplets, also known as dots, in a point-like and matricial fashion.

In practice, a few hundred to a few thousand droplets are used per chip. An analyte to be studied, which may, for example, contain fluorescently labelled target molecules, is then pumped over this surface. This generally results in different chemical (binding) reactions between the target molecules contained in the analyte and the fixed or immobilized capture molecules. As mentioned above, the target molecules are labelled with dye molecule components, usually fluorochromes, in order to observe these reactions or bindings. The presence and the intensity of light which is emitted from the fluorochromes provides information about the progress of the reaction or binding in the individual droplets on the substrate so that it is possible to draw conclusions about the presence and/or the property of the target molecules and/or capture molecules. When the corresponding fluorescently labelled target molecules of the analyte react with and, respectively, bind to the capture molecules immobilized on the surface of the support substrate, this reaction and binding, respectively, can be detected by optical excitation with a laser and measurement of the corresponding fluorescence signal.

Substrates with a high but defined porosity have several advantages over planar substrates as a basis for such biochips. More detection reactions can take place on the greatly enlarged surface area. This increases the detection sensitivity for biological assays. The target molecules dissolved in the analyte are brought into close spatial contact with the surface of the substrate (<10 μm) by pumping them through the channels between the front and back sides of the porous substrate. On this size scale, diffusion is a very effective transport process which covers the distances between a target molecule to be detected and the capture molecule immobilized on the surface within a short period of time. This may increase the rate of the binding reaction and thus distinctly shorten the duration of the detection method.

An example of a substrate having a defined porosity of this type is electrochemically produced porous silicon (cf. DE 42 02 454, EP 0 553 465 or DE 198 20 756).

Many of the analytical methods currently used in drug research and clinical diagnostics employ optical methods for the detection of binding events between the substance to be detected and capture molecules (e.g. DNA hybridizations, antibody-antigen interactions and protein interactions). The substance to be detected is in this case provided with a marker which fluoresces after excitation with light of a suitable wavelength (fluorescence method) or which causes a chemical reaction that in turn generates light (chemiluminescence method). If the substance to be detected, i.e. the target molecule, binds with the immobilized capture molecule on the surface, then this may be detected optically, for example via luminescence. The term “luminescence” here refers to the spontaneous emission of photons in the ultraviolet to infrared spectral range. Luminescence excitation mechanisms may be optical or non-optical in nature, for example electrical, chemical, biochemical and/or thermal excitation processes. Thus, in particular, chemi-, bio- and electro-luminescence and also fluorescence and phosphorescence are intended to be covered by the term “luminescence” for the purposes of the present invention.

Porous substrates with a high optical density and low reflectivity, such as, for example, porous silicon whose reflectivity is 50 to 70% in the visible range of the spectrum, however, do not produce the expected results in conjunction with fluorescence or chemiluminescence methods in so far as the experimentally observed light-signal yield falls far short of the theoretically attainable values. The reason for the, compared to the theoretical values, reduced experimentally observed light-signal yield when such porous substances are used lies, on the one hand, in problems with the fluorescence emission of the substance or binding to be studied and, on the other hand, in the case of a fluorescence method, in problems with the optical fluorescence excitation.

If (luminescence) light is generated throughout the volume of the pores, then the reflectivity of the pore walls plays a decisive part in effectively guiding the optical signal to the surface. In the case of chemiluminescence, the light signal is radiated isotropically in all spatial directions. Consequently, only a very small proportion of the light generated radiates directly within the aperture angle of the individual pore. All other optical paths are reflected multiple times by the walls of the pores before they reach the opening of the pore in question. Even with reflectivities which are only a little less than 100%, however, the intensity of a signal will be greatly reduced after multiple reflections. As a consequence of this effect, this proportion of the generated signal will be greatly attenuated on its path out of the pore and can then scarcely make any contribution to the overall signal.

Attenuation due to multiple reflections by the pore walls, which has already been described in connection with the problems of fluorescence excitation, also constitutes a serious problem for the emission of luminescence. Only fluorophores (fluorescent substances in the analyte) which radiate directly in the direction of the pore opening are available in unattenuated form for a fluorescent signal. All the other optical paths are reflected at least once by the walls of the pores before they reach the opening of the pore. Even with reflectivities which are only a little less than 100%, these multiple reflections will lead to a significant attenuation of the optical signal to be detected.

In order to resolve the aforementioned problems of intensity attenuation due to multiple reflections, it has been proposed to arrange reflective layers on the pore walls to reduce the reflection losses, thereby enabling the excitation and emission light to be guided out of the pores in an improved manner. However, this solution approach was unable to produce any significant improvement in the signal yield.

### SUMMARY OF THE INVENTION

It is therefore an object of the present invention to provide a device or “biochip base module” for the detection of biochemical reactions and/or bindings, which device or module is intended to deliver a higher absolute signal yield with, at the same time, low background signal in the context of analytical methods based on fluorescence or chemiluminescence, so as to increase the detection sensitivity of tests to be carried out with the final biochip. Another object of the invention is to give a method of detecting biochemical reactions and/or bindings which allows a higher absolute signal yield with an improved signal-to-noise ratio. Finally, it is an object of the invention to give a preparation method for a preferred device of the invention.

According to the invention, a device comprises a two-dimensionally formed support material which has, spread across at least one surface area, a multiplicity of pores which stretch throughout from one surface of the support material to the opposite surface, with
the pores being bounded in each case by a pore boundary area of the pore walls formed in the support material along the particular longitudinal axes of the pores,
at least part of the pore walls having at least in some sections a layered structure containing a first layer forming the pore boundary area and a second layer adjacent to the first layer and spaced apart from the pore boundary area, and
the refractive index n_{waveguide} of the first layer being greater than the refractive index n₂ of the second layer.

The support material is thus interspersed with a multiplicity of pores which extend in the support material and allow, for example, a liquid analyte to pass from one of the surfaces of the support material to the opposite surface. The pores are bounded along their longitudinal axes by pore boundary areas which are located in the pore walls in the support material. The pore boundary areas thus constitute the outside surfaces of the pore walls, i.e. the interfaces between the support material and the pores. The pore walls are that proportion by volume of the support material which extends between the pores so that the pores are separated from one another by the pore walls.

The invention proposes to construct at least some sections of at least part of the pore walls as a layered structure. A first layer which forms the pore boundary area (pore-support material interface) is here adjacent to a second layer which is spaced apart from the pore boundary area. This second layer is further away from the central axis of the pore than the first layer. If the pores, for example, have an essentially rectangular cross section in a sectional plane which runs parallel to the surface plane of the support material, the first layer, in cross-sectional view, has preferably an essentially frame-like shape. The second layer may be designed, for example, concentrically with respect to the first layer. If the pores have a rectangular-cylindrical or circular-cylindrical shape, then the first layer is preferably in the shape of a hollow cylinder whose inner diameter corresponds to the pore diameter.

The thickness of the first layer is preferably in a range from 50 nm to 1000 nm, preferably 50 nm to 500 nm. The second layer may, for example, be part of the support material itself or be arranged between a core of the support material and the first layer. In the latter case, the thickness of the second layer is preferably at least 1 μm. Preferably, all pores of the support material have such a layered structure of the pore walls in the region of the pore boundary areas. Preference is given to the layered structure extending over the entire length of the pores.

It is possible to define a waveguide in the pore walls close to the pore boundary areas by the layered structure described, which has a first layer having a refractive index n_{waveguide} which is greater than the refractive index n₂ of the second layer. For if an analyte (liquid or gaseous) is introduced into the pores, whose refractive index nₚₒᵣₑ is likewise less than the refractive index n_{waveguide} of the first layer, then an optical signal may be carried in the first layer in the form of a waveguide mode. For example, starting from one of the surfaces of the support material, excitation light may be coupled into the first layer which forms the waveguide core and which has the refractive index n_{waveguide}, and is then, owing to the limitation by layers with smaller refractive index n₂, nₚₒᵣₑ<n_{waveguide}), carried in the form of a waveguide mode in the first layer.

An adhesion promoter layer which is transparent for the wavelength range of interest and which preferably has a thickness of ≦20 nm may be arranged, where appropriate in some sections, between the first layer and the second layer, improving, in particular, the mechanical connection between the first layer and the second layer.

The optical properties of the layer material of the first layer are preferably adapted to the wavelength of the excitation light to be coupled in. The first layer is preferably transparent in a predetermined spectral range or the spectral range of interest. The spectral range may constitute, in particular, the visible, ultraviolet or infrared spectral range. The waveguide mode carried in this way in the first layer has an evanescent field which depends on the differences in the refractive indices and the waveguide geometry and which extends into the pores, in particular in a region close to the pore boundary areas. This evanescent field decays exponentially as a function of the distance to the pore boundary area and may be utilized to optically excite fluorescent substances which are arranged at the pore boundary area or in the immediate proximity thereof (penetration depth of the evanescent field: up to about 1 μm).

The problems with fluorescence excitation, described at the outset, can be avoided effectively by introducing the excitation light, for example for excitation of fluorochromes, into the pore by efficiently carrying it in a waveguide mode with low losses rather than by means of multiple reflections on the pore walls with high losses. The waveguide concept is equally useful also in the case of the emission of luminescence (both in fluorescence and in chemiluminescence methods). If, for example, the substance to be detected emits with the immobilized capture molecule on the pore boundary area, i.e. in the evanescent field region of the waveguide mode, a photon, then the latter may be coupled out of the device with low losses and led towards a detector. The attenuation, described at the outset, due to multiple reflections on imperfectly reflecting pore walls does not occur. Thus it is possible to markedly increase the absolute signal yield and to achieve a lasting improvement of the signal-to-noise ratio.

According to a preferred embodiment, the layered structure has a third layer adjacent to the second layer and spaced apart from the first layer.

The first layer preferably comprises Ta₂O₅, HfO₂, Y₂O₃, Al₂O₃, Nb₂O₅, Si₃N₄, TiO₂, TaO₂ and/or nitride or oxynitride of Al, Si or Hf, and the second layer comprises glass, plastic, in particular organic or inorganic polymers, transparent dielectrics and/or SiO₂. The materials of the first and second layers are selected in such a way that the refractive index n_{waveguide} of the first layer is always greater than the refractive index n₂ of the second layer. Furthermore, the analyte which is operationally introduced into the pores is selected with respect to its refractive index nₚₒᵣₑ in such a way that n_{waveguide}>nₚₒᵣₑ.

The third layer preferably comprises metals, semiconductors and/or plastic, in particular organic or inorganic polymers. The third layer may, in particular, be designed in a reflecting and/or non-transparent manner, in order to divide, for example a multiplicity of pores into groups by forming regions or compartments, with “optical cross-talk” between the groups being prevented by the reflecting walls. The third layer preferably comprises silicon which may be n-doped or p-doped.

According to a preferred embodiment, the pore diameter in a region close to the surface of the support material increases towards the surface of the support material. The region of the pores which is close to the surface has preferably a funnel-like or conical shape, it being advantageous for the pore diameter to continually increase towards the surface of the support material. The first layer is preferably guided in the conical region of the pores up to the surface of the support material. This arrangement allows excitation light to be particularly efficiently coupled into the first layer which constitutes the waveguide core. Such an arrangement of the pores or pore walls in the region of the surface of the support material equally also improves coupling out of luminescence light to be detected which was guided in the first layer to the surface of the support material.

Preference is given to the pores being essentially cylindrical, in particular rectangular, circular, slit-like or elliptical.

According to another embodiment, the first layer has scattering centres and/or defects. Such a specific incorporation of scattering centres/defects at the interface of the first layer and/or in the first layer itself, which may be carried out, for example, by roughening or doping, may improve coupling-in of excitation light and, respectively, coupling-out of emitted light.

The pores preferably have a pore diameter in the range from 500 nm to 100 μm. The support material preferably has a thickness of between 100 and 5000 μm. The pore density is preferably in the range from 10⁴ to 10⁸/cm².

According to a preferred embodiment, the support material has a superstructure made of a material which is non-transparent and/or reflects in the predetermined spectral range. The superstructure may have any form. The superstructure preferably is an essentially cylinder-shaped frame which stretches from the one surface to the opposite surface of the support material and which includes at least one of the pores. The frame divides the support material into regions or compartments. The frame is preferably made of a reflective or non-transparent material so as to prevent “optical cross-talk” between the individual regions. The frame may also be in the form of a partial frame with one or more open sides. The cylinder axis of the cylindrical frame is preferably arranged parallel to the longitudinal axes of the pores. The cylinder axis of the frame and the longitudinal axes of the pores are preferably perpendicular to the support material surfaces facing each other. The frame preferably has a silicone core.

According to a preferred embodiment, capture molecules or probes, selected from the group consisting of DNA, proteins and ligands, are covalently bound to at least some sections of the pore boundary area of at least one of the pores. The capture molecules are preferably oligonucleotide probes which are bound via terminal amino or thiol groups to linker molecules which in turn are bound via covalent and/or ionic groups to the pore boundary area. The linker molecules are usually based on a bifunctional organosilicon compound. Possible examples of such bifunctional organosilicon compounds are alkoxysilane compounds having one or more terminal functional groups selected from epoxy, glycidyl, chloro, mercapto or amino. The alkoxysilane compound is preferably a glycidoxyalkylalkoxysilane such as, for example, 3-glycidoxypropyltrimethoxysilane, a mercaptoalkylalkoxysilane such as, for example, γ-mercaptopropyltrimethoxysilane, or an aminoalkylalkoxysilane such as, for example, N-β-(aminoethyl) γ-aminopropyltrimethoxysilane. In this context, the length of the alkylene radicals acting as spacers between the functional group such as, for example, epoxy or glycidoxy, which binds with the actual capture molecule or probe, and the trialkoxysilane group is not subject to any restriction. Spacers of this type may also be polyethylene glycol radicals. The oligonucleotides usable as capture molecules by way of example may be prepared using the synthesis strategy as described in Tet. Let. 22, 1981, pages 1859 to 1862. The oligonucleotides may be derivatized with terminal amino groups either at the 5′ or the 3′ terminals during the preparation process. Capture molecules of this type may also be attached by first treating the pore boundary areas or the first layer with a chlorine source such as Cl₂, SOCl₂, COCl₂ or (COCl)₂, where appropriate by using a free radical starter such as peroxides, azo compounds or Bu₃SnH, followed by a reaction with an appropriate nucleophilic compound such as, in particular, with oligonucleotides or DNA molecules having terminal primary amino groups or thiol groups (see WO 00/33976). If the first layer used within the framework of the devices of the invention is an Si₃N₄ layer, then the latter may, for example on their surface layer, be converted into an oxynitride layer in order to covalently attach corresponding capture molecules via linker molecules.

Attaching, via such linker molecules, capture molecules selected from among DNA, proteins and ligands, preferably oligonucleotides having terminal amino or thiol groups, results in a usable, finished biochip. Such a device of the invention may have the function of a 96-sample support with the density of a microarray. Furthermore, microchip technologies available in the prior art may be parallelized based on such devices of the invention.

According to the invention, the use of any of the inventive devices described above is used as a basis for a sample support in methods of detecting biochemical reactions and/or bindings and also, to this end in particular, for studying enzymic reactions, nucleic acid hybridizations, protein-protein interactions and protein-ligand interactions.

According to another aspect of the invention, a method of detecting chemical or biochemical reactions and/or bindings is proposed, which comprises the steps of:
providing a device or biochips of the invention;
introducing a substance to be studied into at least one of the pores of the support material, with the refractive index nₚₒᵣₑ of the substance to be studied being less than the refractive index n_{waveguide} of the first layer;
coupling out luminescence light of the substance to be studied; and
studying the luminescence light of the material to be studied.

The material to be studied here may be gas-like or liquid-like. Since the refractive index n_{waveguide} of the first layer is greater than the refractive indices n₂ of the second layer and nₚₒᵣₑ of the analyte-filled pore, it is possible to guide excitation light or emission light to be detected from the evanescent field region as waveguide modes in the first layer. This allows efficient coupling-in or ciupling-out of the optical signal, since no attenuation with high losses due to multiple reflections by the imperfectly reflecting pore walls occurs. In this way, a sustained improvement of the absolute signal yield and an improvement in the attainable signal-to-noise ratio can be achieved.

The method preferably comprises, in addition, the step of coupling excitation light as waveguide modes into the first layer of the layered structure in order to excite the material to be studied on the pore boundary area in an evanescent field of the waveguide modes. As has already been discussed in detail above, it is possible, for example, to excite fluorophores at the pore boundary area via the evanescent field of the modes guided in the waveguide, without a volume excitation taking place. This approach makes it possible to improve the excitation of fluorophores on the pore boundary area of the waveguide many times compared to the customarily used volume excitations.

According to another aspect of the invention, a method of detecting chemical or biochemical reactions and/or bindings comprises the steps of:
providing a device or biochips of the invention;
introducing a substance to be studied into at least one of the pores of the support material, with the refractive index nₚₒᵣₑ of the substance to be studied being less than the refractive index n_{waveguide} of the first layer;
coupling excitation light as waveguide modes into the first layer of the layered structure in order to excite the material to be studied at the pore boundary area in an evanescent field of the waveguide modes; and
studying the excitation light coupled out of the first layer, after interaction with the material to be studied.

Thus the chemical or biochemical reactions and/or bindings are not detected by studying a possibly emitted luminescence of the material to be studied (analyte to be studied). Instead, the reactions and/or binding events of the material to be studied are detected by way of their influence on the modes guided in the waveguide. Thus, the material or chemical processes to be studied can be detected in a “label-free” manner.

According to a preferred variant, this may take place due to a change in the refractive index in the immediate proximity of the pore boundary area, which change is caused by molecules of the analyte attaching or binding to the capture molecules on the pore boundary area of the first layer. This change in the refractive index detunes the parameters of the modes guided in the waveguide, leading to a coupling via the evanescent field of the waveguide modes. This detuning may be detected, for example, by a comparative measurement of the waveguide modes before and after attachment or binding of the molecules.

The detection may be carried out by way of example as follows:
(a) A reduction or increase in attenuation of the excited modes in the waveguide may be observed, with the geometry of the coupling-in of light remaining the same. The attenuation of modes may be measured by way of the change in light intensity, for example at the exit of the waveguides.
(b) The geometry of the coupling-in of light is continually altered and the attenuation of the waveguide modes is recorded. From this course, the change in the refractive index on the waveguide surface or pore boundary area with capture molecules immobilized thereto (and thus the binding event) can be detected (preferably again by way of a comparative measurement before and after attachment of the molecules).

According to another preferred variant, it is possible for the attachment of molecules absorbing in a specific wavelength range to the surface of the waveguide via the immobilized capture molecules to draw energy via the evanescent field from the modes guided in the waveguide (due to adsorption of the attached molecules). The molecules are adsorbed to the pore boundary area which has been provided with the capture molecules.

For example, the detection may successfully be carried out by way of the reduction or increase in attenuation of the excited modes in the waveguide, with the geometry of the coupling-in of light remaining the same. The attenuation of the modes may be measured by way of the change in the light intensity in the waveguide, for example at the exit.

According to another aspect of the invention, a method of controlling chemical or biochemical reactions or syntheses comprises the steps of:
providing a device or biochips of the invention;
introducing a synthetic substance into at least one of the pores of the support material, with the refractive index nₚₒᵣₑ of the synthetic substance being less than the refractive index n_{waveguide} of the first layer; and
coupling light as waveguide modes into the first layer of the layered structure in order to optically excite at least the synthetic substance at the pore boundary area in an evanescent field of the waveguide modes.

The devices of the invention are particularly suitable for the locally limited, light-controlled synthesis of molecules on the pore boundary areas of the pore walls. For example, EP 0 619 321 and EP 0 476 014 describe the method of light-controlled synthesis for planar substrates. Full reference is made to the disclosure of these documents with respect to the structure and light-controlled synthesis method so that, to this extent, these documents also form part of the disclosure of the present application. The efficient propagation of the light into the pores (via the waveguides) may drive or control, via the evanescent field, photochemical reactions on the pore boundary areas of the pore walls. In particular, it is possible to carry out in this way complex sequential light-controlled chemical reactions on the pore boundary areas.

Optical cross-talk between the individual pores or regions/compartments is stopped by reflective/adsorptive walls. This solves a major problem with light-controlled synthesis on planar substrates.

According to another aspect of the invention, a method of preparing a device of the invention is proposed, which comprises the following steps:
(a) providing a two-dimensional support material made of silicon and having two surfaces facing each other;
(b) generating blind holes whose depth is less than the thickness of the support material by electro-chemically etching one of the surfaces of the support material;
(c) removing the support material from the opposite surface at least to the bottom of the blind holes to obtain pores which stretch throughout from the one surface to the opposite surface of the support material;
(d) subjecting the support material obtained in step (c) to an oxidation so that the pore walls of the support material consist, at least in the region close to the pores, of SiO₂ to form the second layer; and
(e) coating at least one of the pore walls in order to form the first layer.

This preferred preparation method can be used to prepare a particularly preferred embodiment of a device of the invention. The starting point here is preferably a monocrystalline silicon substrate which may be n-doped, for example.

Prior to etching of the support material, a mask layer is preferably arranged at least in some regions on the one surface of the support material and on the inner surfaces of the blind holes generated in step (b), with the mask layer being removed after the etching step.

Preferably, the blind holes are generated in such a form that the distance between the blind holes which are designed in an otherwise essentially regular arrangement is changed in some sections with the formation of inter-region transitions with increased silicon wall thickness, with the thickness of the silicon walls between the inter-region transitions being greater than the thickness of the silicon walls inside the regions by the amount of the increased distance between the blind holes.

The support material etched in step (c) is preferably subjected to a thermal oxidation in such a way that, as a function of the silicon wall thickness, the regions with thinner silicon walls are fully oxidized while, in the case of the inter-region transitions with an increased wall thickness, the silicon walls are not fully oxidized so that a silicon core remains in the walls.

In step (b) of the method of the invention electro-chemical etching into the silicon is carried out. Such a method is disclosed, for example, in EP 0 296 348, EP 0 645 621, WO 99/25026, DE 42 02 454, EP 0 553 465 or DE 198 20 756, to which reference is made in full scope and the disclosure of which is, to this extent, intended to be part of the present application. In the course of such electrochemical etching, blind holes or pores with aspect ratios of, for example, 1 to 300 or more may be etched in an essentially regular arrangement in silicon. Since, with suitably chosen parameters, the electrochemical pore-etching method allows the pore spacing (pitch) to be altered within particular limits, the thickness of the resulting silicon walls can be locally altered by changing the pore spacing or omitting an entire row of pores in the otherwise regular arrangement of blind holes or pores.

In order to obtain pores which extend across the support material or substrate (Si wafer) and are open on both surfaces of the support material, silicon is eroded on the rear side of the Si wafer, for example by KOH etching, after having etching the blind holes, while the front side of the wafer and the inside of the blind holes or pores are protected by a mask layer such as, for example, a silicon nitride layer produced by CVD deposition with a thickness of 100 nm, for example. The mask layer may then be removed by means of HF treatment, for example. Sputtering, laser ablation and/or polishing processes, for example a CMP process, are equally suitable for the rear-side erosion of the Si wafer.

This generates a silicon wafer or silicon support material which is matricially provided with regular pores which constitute through-tubes which connect the front and rear sides of the wafer with one another.

The diameter of these pores may be enlarged or widened after their preparation, for example by etching in KOH. If Si(100) is used as a starting material, then essentially square pores are obtained by such etching, owing to the crystal structure. For example, assuming a pore diameter of approx. 5 μm with a distance of 12 μm between the mid-points of two pores (pitch), it is possible to enlarge in this way the pore diameter from, for example, 5 μm to 10 to 11 μm. At the same time, the thickness of the silicon walls between the pores is reduced to 2 to 1 μm. In this way, a more or less square lattice of thin silicon walls is obtained. The depth of the pores and, respectively, the length of the silicon walls here correspond to the original thickness of the silicon wafer less the thickness of the Si layer on the rear side when opening the pores.

In the oxidation step (d), the lattice obtained in this way is converted into SiO₂ in a thermal oxidation process, for example at a temperature of 1100° C. and with a duration of 6 hours, by oxidation as a function of the particular pore-wall thickness. The structure of the substrate remains essentially unchanged in the process, apart from a volume increase of the wall regions due to the oxidation of Si to SiO₂.

If, in step (b) the distance between the blind holes or pores is increased periodically, for example every 5, 10 or 20 pores, and slightly, for example by 1 μm, then this produces a superstructure which is composed of regions with arrays of pores (for example 5×5, 10×10, 20×20). The thickness of the silicon walls between these regions is greater than the thickness of the silicon walls inside the regions by the amount of the increased pore spacing. A subsequent oxidation fully oxidizes the regions with thin silicon walls to SiO₂. However, the silicon walls of the transitions between the regions, which have an increased wall thickness, are not completely oxidized so that a silicon core remains in the walls, with the core of silicon turning into silicon dioxide over the cross section towards the outside of the walls forming the frame. This produces locally completely transparent regions of SiO₂ which are separated from one another by non-transparent walls containing a silicon core.

The waveguide, i.e. the first layer, is preferably prepared using a CVD process. However, it is also possible to employ sputtering, vapour deposition or wet-chemical assembly steps. In contrast to sputtering or vapour deposition processes, it is possible to obtain with the aid of a CVD method a homogeneous deposition of the waveguide material (of the layered structure) over the entire length of the pores, despite the large aspect ratio. If the waveguide material used is Si₃N₄, the latter may be deposited, for example, from DCS (dichlorosilane) and NH₃ as precursors in a reactor at a temperature of 650° C. and a pressure of 400 mTorr, for example.

Since the waveguide material is deposited over the whole area, it may, if required, subsequently be removed again from the planar surfaces of the silicon substrate. This is possible using, for example, a CMP, sputtering or etching process. Plasma etching is also suitable by way of example, which, in particular, enables the preferred funnel-like structure of the waveguide in the pore openings to be generated.

According to another aspect, the invention proposes a method of detecting chemical or biochemical reactions and/or bindings, which method comprises the steps of:
providing a device having a two-dimensionally formed support material which has, spread across at least one surface area, a multiplicity of pores which stretch throughout from one surface of the support material to the opposite surface, with the pores being bounded by a pore boundary area of the pore walls formed in the support material along the longitudinal axes of the pores and capture molecules being immobilized thereto, where appropriate via linker molecules;
introducing a substance to be studied into at least one of the pores of the support material, with the refractive index nₚₒᵣₑ of the substance to be studied being greater than the refractive index n_{pore wall} of the pore wall in the region of the pore boundary area;
coupling out luminescence light of the substance to be studied as waveguide modes of the pore forming a waveguide; and
studying the luminescence light of the material to be studied.

In this embodiment, the refractive index nₚₒᵣₑ of the (gaseous or liquid) analyte introduced into the pore is greater than the refractive index n_{pore wall} of the pore walls by which the pores are bounded. Thus the analyte-filled pores are themselves waveguides (waveguide cores) so that the pores are referred to as ‘liquid core waveguides’.

For this embodiment of a waveguide whose core is the analyte-filled pore itself, considerations with respect to variance and geometries of the structure apply, which are analogous to those for deposited waveguides, which have already been described above. For example, the support material may be provided with a further superstructure in order to form, for example, regions/compartments. In order to ensure coupling of light into the waveguides to be as efficient as possible, sharp edges at the openings of the pores should be avoided here as well as in the embodiments described above. The opening region of the pores preferably has a funnel-type structure, the optimal geometry depending on the angular distribution of the excitation light.

The method preferably comprises, in addition, the step of coupling excitation light as waveguide modes into the pore in order to excite the material to be studied.

### BRIEF DESCRIPTION OF THE DRAWINGS

The present invention is described by way of example below, with reference to the accompanying drawings of preferred embodiments in which:

FIGS. 1(A) to 1(C) depict schematic cross-sectional views of preferred embodiments of devices of the invention, with the cross-sectional plane running along the longitudinal axes of the pores;

FIG. 2(A) depicts a diagrammatic plan view of a preferred embodiment of a device of the invention;

FIG. 2(B) depicts a schematic cross-sectional view of the embodiment depicted in FIG. 2(A), with the sectional plane running parallel to the longitudinal axes of the pores;

FIGS. 3(A) to 3(E) depict a schematic sectional view of possible pore embodiments;

FIG. 4(A) depicts a schematic plan view of a preferred embodiment of a liquid core waveguide;

FIG. 4(B) depicts a schematic cross-sectional view of the embodiments depicted in FIG. 4(A), with the cross-sectional plane running parallel to the longitudinal axes of the pores; and

FIGS. 5(A) to 5(C) depict preferred embodiments of ore geometries for a liquid core waveguide.

### DETAILED DESCRIPTION OF THE PREFERRED MODE OF THE INVENTION

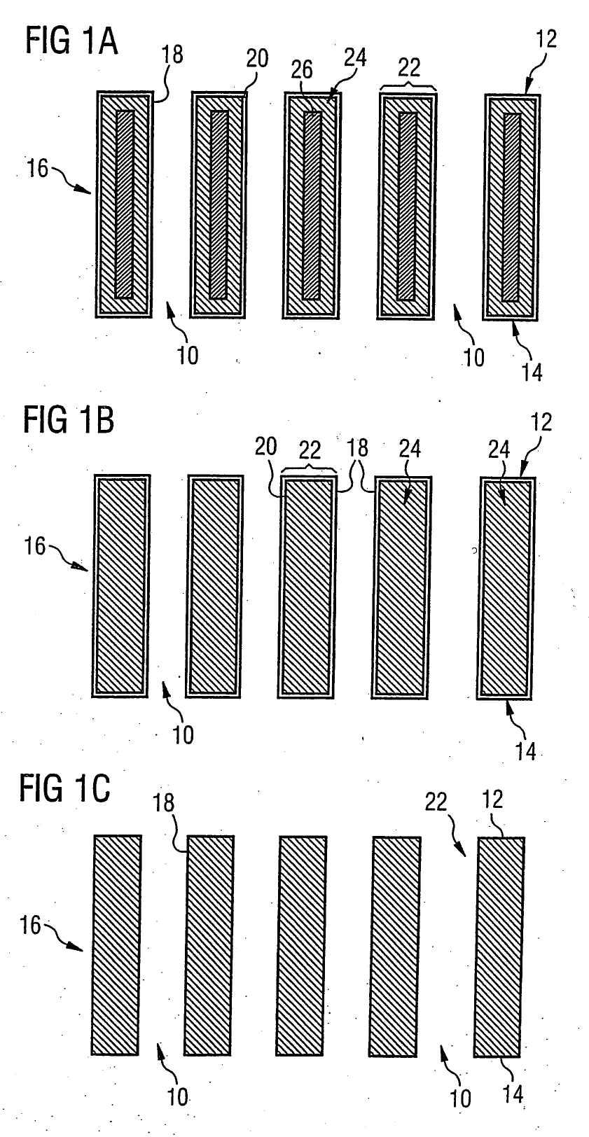

FIG. 1(A) depicts in a schematic sectional view a preferred embodiment of a device of the invention. The sectional plane of the highly schematic sectional view runs along the longitudinal axes of four pores 10 which are depicted by way of example and which stretch throughout from one surface 12 of the two-dimensionally formed support material 16 to the opposite surface 14. In this embodiment, the pores 10 are cylinder-shaped, with the cylinder axes of the pores 10 coinciding with the surface normals of the two-dimensional support material 16. In operation, an analyte may be passed from the one surface 12 of the support material 16 through the pores 10 to the opposite surface 14 in order to study, for example, biochemical reactions and/or bindings.

The form of a pore 10 is in each case defined by a pore boundary area 18 which, in this case, gives each of the pores 10 a cylinder-like shape. The end faces of the cylinders which define the pores 10 are open so that an analyte may, for example, be introduced from the surface 12 and leave the pores 10 at the surface 14. The pore boundary area 18 by which the pore 10 is bounded along its longitudinal axis represents the outer surface of a first layer 20 which is part of the pore wall 22. The pore walls 22 are that proportion by volume of the support material which extends between the pores 10. Thus the pore walls 22 separate the pores 10 from one another. The pore boundary areas 18 are thus the particular interfaces between the pore walls 22 and the inside of the pores 10.

In the embodiment depicted in FIG. 1(A), all pore walls 22 have, over their entire length, a layered structure whose outermost layer is the first layer 20. The first layer 20 is arranged next to a second layer 24 which is spaced apart from the assigned pore 10. In the present embodiment, the second layer 24 is separated from the particular pore 10 by the first layer 20.

While in the embodiment depicted in FIG. 1(B) the pore walls 22 consist of the first layer 20 and the second layer 24, with the second layer 24 filling the entire core of the pore walls 22 or of the support material 16, the embodiment depicted in FIG. 1(A) provides a third layer 26 which preferably constitutes the core region of the pore walls 22.

The refractive index n_{waveguide} of the first layer 20 is greater than the refractive index n₂ of the second layer 24. In operation, an analyte is introduced into the pores 10, whose refractive index nₚₒᵣₑ is likewise less than the refractive index n_{waveguide} of the first layer 20. The first layer 20 of the layered structure of the pore walls 22 is thus surrounded by the second layer 24 and by the analyte in the pores 10, each of which have a smaller refractive index. Constructing the layers in this way with suitably chosen refractive indices enables an electromagnetic wave to propagate in the form of waveguide modes in the first layer 20, the direction of propagation being parallel to the longitudinal axes of the pores. The waveguide modes (main mode and modes of higher order) are thus guided in the region close to the pores 10 in the first layer 20. Although the intensity of the electromagnetic wave within the first layer 20 is greater than in the adjacent layers in such an arrangement, an evanescent electromagnetic field of the electromagnetic wave enters, in particular, the space adjacent to the pore boundary area 18 inside the pore 10. This evanescent field decays exponentially in a direction which is perpendicular to the longitudinal axis of the waveguide.

When an optical excitation signal is introduced into the first layer 20, then it is possible for substances to be studied which are located at or in the immediate proximity of the assigned pore boundary area 18 to be optically excited by the evanescent field of the modes guided in the waveguide 20. Conversely, photons which are emitted in the evanescent field region by the material to be studied may leave the support material 16 as guided waveguide modes via the first layer 20.

Consequently, the embodiments depicted in FIGS. 1(A) and 1(B) are not subject to the principle of attenuation due to multiple reflections on imperfectly reflecting pore walls, which principle is known from the prior art. Instead, excitation light may be introduced into and guided in the first layer 20 as waveguide modes with low losses. The optical signal to be detected may, as a waveguide mode in the first layer 20, be coupled out of the support material 16 and directed towards a detector.

Excitation light may be coupled into the first layer 20 by using one or more randomly arranged excitation light sources, where appropriate in combination with diffractive optical elements. The excitation light may be irradiated simultaneously or sequentially from various directions with identical or different wavelengths. The light sources may be coherent or non-coherent light sources which are superimposed coherently or non-coherently. The excitation may be carried out by way of a hologram, for example.

The luminescence is detected using at least one detector, for example CCD cameras, photodiode arrays, avalanche photodiode arrays, multichannel plates and/or multichannel photomultipliers.

The layers of the embodiment depicted in FIG. 1(A) (four-component embodiment) may, for example, be chosen as follows:
Main component of the analyte located in the pores 10 (refractive index nₚₒᵣₑ): air, H₂O, including aqueous buffer solutions which may contain, where appropriate, small proportions of organic solvents, glycerol;
First layer 20 (refractive index n_{waveguide}): Ta₂O₅, HfO₂, Y₂O₃, Al₂O₃, Nb₂O₅, Si₃N₄, TiO₂, TaO₂ and/or nitride or oxynitride of Al, Si or Hf;
Second layer 24 (refractive index n₂): glass, plastic, transparent dielectrics and/or SiO₂; and
Third layer 26 (refractive index n₃): metals, semi-conductors, in particular silicon (undoped, p-doped or n-doped), plastic, in particular inorganic or organic polymers.

The materials of the layers for the embodiment depicted in FIG. 1(B) (three-component embodiment) may, for example, be chosen as follows:
Main component of the analyte located in the pores 10 (refractive index nₚₒᵣₑ): air, H₂O, including aqueous buffer solutions which may contain, where appropriate, small proportions of organic solvents, glycerol;
First layer 20 (refractive index n_{waveguide}): Ta₂O₅, HfO₂, Y₂O₃, Al₂O₃, Nb₂O₅, Si₃N₄, TiO₂, TaO₂ and/or nitride or oxynitride of Al, Si or Hf; and
Second layer 24 (refractive index n₂): glass, plastic, transparent dielectrics and/or SiO₂.

A targeted incorporation of scattering centres or defects on the pore boundary areas 18 and/or in the waveguides themselves, i.e. in the first layer 20, may be advantageous. These scattering centres or defects may be carried out, for example, by roughening the pore boundary areas 18 or by specifically doping the first layer 20. Such a measure may improve the coupling in and out of excitation light.

FIG. 1(C) depicts another embodiment of the invention. In the embodiment depicted in FIG. 1(C), the pores 10 themselves are utilized as optical waveguide. The pores 10 thus constitute a “liquid core waveguide”. This is possible due to the fact that for the refractive indices of the pore walls 22 and of the analyte operationally contained in the pores 10: nₚₒᵣₑ>n_{pore wall}, where n_{pore wall} is the refractive index of the pore walls 22 of the support material 16.

In contrast to the embodiments depicted in connection with FIGS. 1(A) and 1(B), in which the waveguide is formed in the pore walls, the pore 10 itself constitutes the waveguide in the embodiment depicted in FIG. 1(C). Excitation light and, respectively, emitted photons of the material to be studied may leave the support material 16 in the form of waveguide modes propagating with low losses, without the attenuation mechanism described at the outset resulting in substantial signal losses due to multiple reflections.

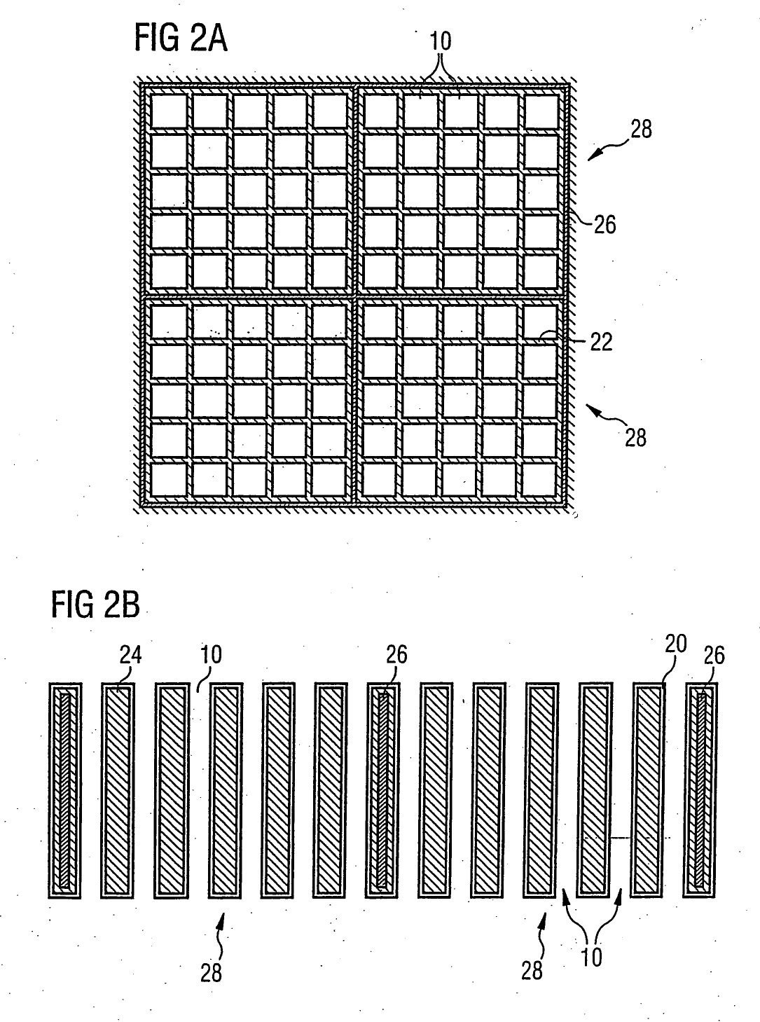

FIG. 2(A) depicts in plan view a particularly preferred embodiment of a device of the invention. In this embodiment, the support material 16 which is regularly interspersed with pores 10 has additionally been subjected to a superstructuring in order to generate a further superstructure. The support material 16 is divided into small regions or compartments 28 which in each case comprise at least one of the pores 10. The regions 28, four of which are depicted in FIG. 2(A) by way of example, are preferably separated from adjacent regions 28 with the aid of frame-like walls made of a preferably reflective material.

As has been described in connection with FIG. 1(A), the pore walls 22 may have a third layer 26 which may be used for constructing the reflective walls. For example, the reflective frame-like walls may be constructed from metal or semiconductors. Within the regions 28, the pore walls 22 between the pores 10 consist preferably of a material which is transparent for wavelengths in the predetermined spectral range or spectral range of interest. This geometry efficiently suppresses the propagation of scattered light and cross-talking of an optical signal from one region 28 to an adjacent region 28.

FIG. 2(B) depicts the embodiment depicted in FIG. 2(A) in a highly schematic sectional view, with the sectional plane running parallel to the longitudinal axes of the pores 10. In this case, the two-dimensional support material 16 is interspersed with matrixially and regularly arranged pores 10 which in each case have a rectangular cylinder-like form. The superstructure forms a rectangular frame structure, with the frames likewise having a rectangular cylindrical form. The longitudinal axes of the cylindrical frames which define the regions 28 are parallel to the longitudinal axes of the pores 10. In this case, for example, every sixth pore wall 22 between the pores 10 has a core region or a third layer 26 which consists of a reflective material. The remaining pore walls 22 within the region 28 are transparent for wavelengths in the predetermined spectral range.

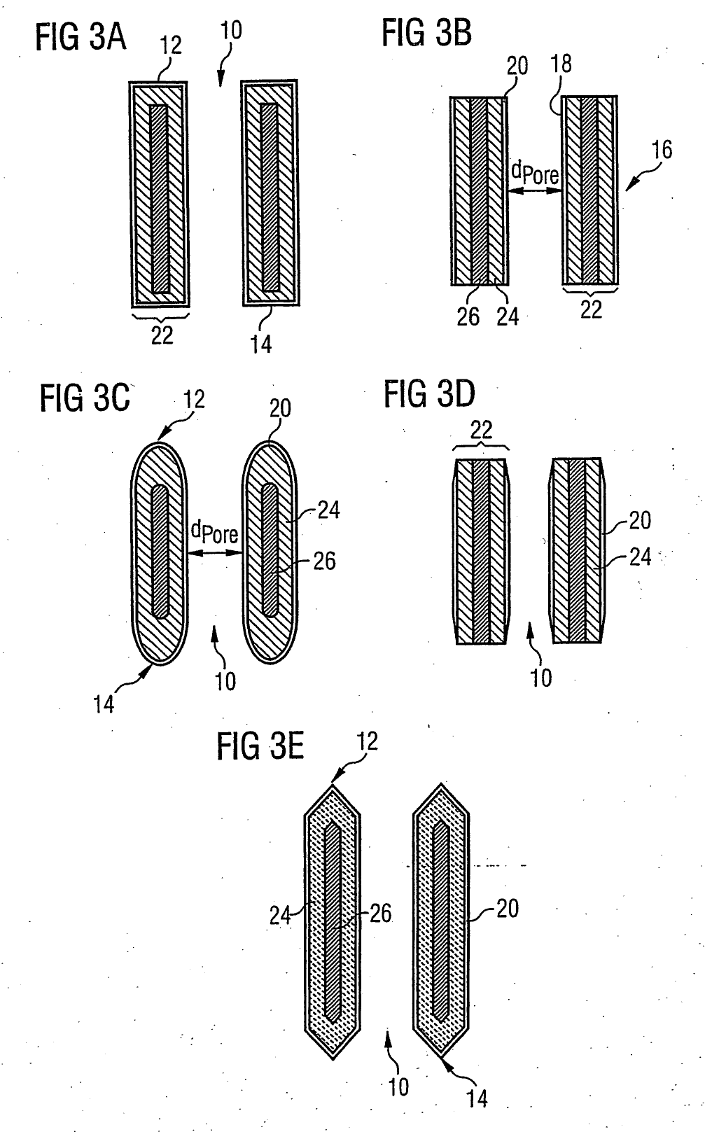

FIGS. 3(A) to (E) depict highly schematic sectional views of preferred pore-wall geometries. In order to ensure that light is coupled into or out of the wave-guides 20 as effectively as possible, a particular design of the pore-wall geometry close to the surfaces 12 or 14 of the support material 16 is advantageous. In particular the variant depicted in FIG. 3(C), in which the diameter dₚₒᵣₑ of the pores 10 increases close to the surfaces 12 and 14 of the support material 16 continually in the direction of these surfaces 12 and 14, has proved to be particularly advantageous. The pores 10 thus have a conical or funnel-type structure in the region close to the surface of the support material 16.

In order to couple light into the first layer 20 as efficiently as possible, the following factors should be taken into consideration:
(1) The higher the proportion of the waveguide structure on the surfaces 12 or 14 of the support material 16 compared to the surface 12, 14 of the support material 16, which has not been provided with the waveguide structure or the structured layer, the higher is the coupled-in proportion of the incident light, which can be expected. This factor is met, in particular, by the embodiments depicted in FIGS. 3(A) and 3(C) to a particular extent.
(2) Sharp edges within the layer structure or the first layer 20 in the inner region of the pores 10 should be avoided.

Particular preference is given to forming a funnel-type structure in the opening region of the pores 10, as is depicted in FIG. 3(C). The exact geometry in the opening region of the pores 10, i.e. the aperture angle, the layer thicknesses and the curvature of the surface, also depends on the angle distribution of the excitation light. However, the embodiments depicted in FIGS. 3(B) and 3(E) are also possible. The variant depicted in FIG. 3(E) is characterized, in particular, by the pyramid-type pore wall regions having, in the region of the surfaces 12 and 14, defined coupling-in areas which are inclined with respect to the plane of the surfaces 12, 14. Thus it is possible to couple in excitation light with an adjustable angle to this coupling-in area. In particular, it is possible in this way to selectively excite only a single waveguide mode. The top side 12 and bottom side 14 of the support material may have different pore-opening geometries.

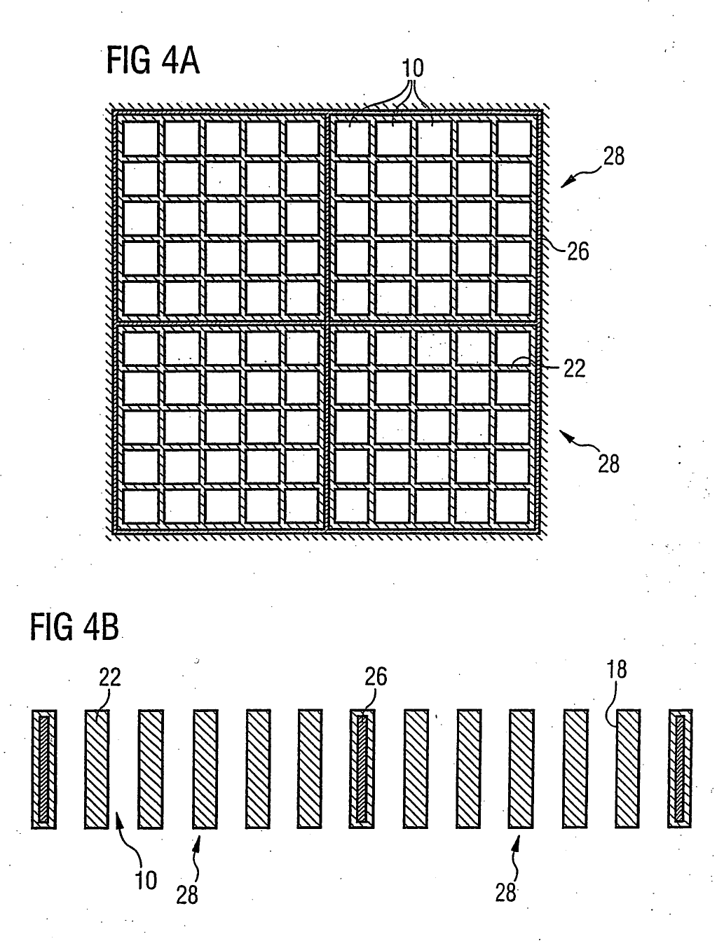

FIG. 4(A), in plan view, and FIG. 4(B), in sectional view, depict another embodiment of the invention. The embodiment depicted in FIG. 4 is an embodiment of the concept described above, in which the pores 10 themselves form the waveguides so that the pore is utilized as a “liquid core waveguide”. In this case too, the support material 16 which is interspersed with a regular arrangement of pores 10, may be subjected to another superstructuring as desired. As has already been described in detail in connection with FIG. 2, this superstructuring may be carried out, for example, by way of dividing a multiplicity of the pores 10 into bounded regions or compartments which are separated from one another by reflective and/or non-transparent walls 26. The frame-like walls 26 by which the regions 28 are bounded may also be designed having at least one open side. The cylinder axes of the preferably cylindrically designed walls 26 of the regions 28 are arranged in such a way that they run parallel to the longitudinal axes of the pores 10.

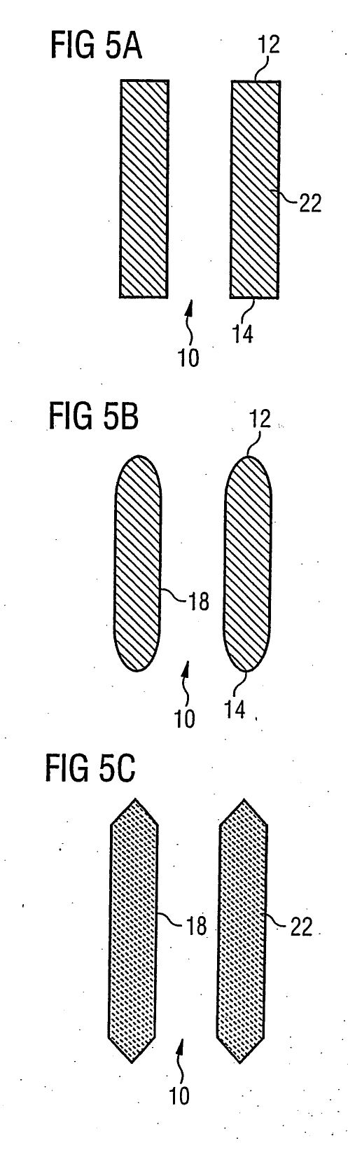

FIGS. 5(A) to 5(C) depict advantageous embodiments of pore geometries for a liquid core waveguide in a highly schematic sectional view. Similarly to the embodiment depicted in connection with FIG. 3(C), a trough- or funnel-type widening of the pores 10 in the region close to the surface of the support material 16 is also advantageous, for the purpose of superior coupling-in and coupling-out of the light, when the pores 10 themselves are utilized as waveguides. FIG. 5(C) corresponds to the variant with defined coupling-in areas, which has already been illustrated in connection with FIG. 3(E).

1. A device comprising:
a two-dimensionally formed support material which has, spread across at least one surface area, a plurality of pores which stretch throughout from one surface of the support material to the opposite surface,
wherein the pores are bound in each case by a pore boundary area of pore walls formed in the support material along particular longitudinal axes of the pores, and at least part of the pore walls have at least in some sections a layered structure containing a first layer forming the pore boundary area and a second layer adjacent to the first layer and spaced apart from the pore boundary area, and
wherein the refractive index n_{waveguide} of the first layer is greater than the refractive index n₂ of the second layer.

2. The device according to claim 1, wherein the first layer is transparent in a predetermined spectral range.

3. The device according to claim 2, wherein the predetermined spectral range is the optically visible, ultraviolet, or infrared spectral range.

4. The device according to claim 1, wherein the layered structure has a third layer adjacent to the second layer and spaced apart from the first layer.

5. The device according to claim 1, wherein the first layer comprises Ta₂O₅, HfO₂, Y₂O₃, Al₂O₃, Nb₂O₅, Si₃N₄, TiO₂, TaO₂ and/or nitride or oxynitride of Al, Si or Hf, and the second layer comprises glass, plastic, transparent dielectrics and/or SiO₂.

6. The device according to claim 4, wherein the third layer comprises metal, semiconductors, and/or plastic.

7. The device according to claim 6, wherein the third layer comprises silicon.

8. The device according to claim 1, wherein the pore diameter in a region close to the surface of the support material increases towards the surface of the support material.

9. The device according to claim 1, wherein the pores are essentially cylinder-shaped or strip-shaped.

10. The device according to claim 1, wherein the first layer has scattering centres and/or defects.

11. The device according to claim 1, wherein the pores have a pore diameter in a range from 500 nm to 100 μm.

12. The device according to claim 1, wherein the support material has a thickness of between 100 and 5000 μm.

13. The device according to claim 1, wherein the density of the pores is in a range from 10⁴ to 10⁸/cm².

14. The device according to claim 1, wherein the support material has at least one superstructure made of a material which is non-transparent and/or reflects in a predetermined spectral range.

15. The device according to claim 14, wherein the superstructure is an essentially cylinder-shaped frame which stretches from the one surface to the opposite surface of the support material and which includes at least one of the pores.

16. The device according to claim 15, wherein the frame comprises at least one core made of silicon.

17. The device according to claim 1, wherein capture molecules selected from the group consisting of DNA, proteins, and ligands are covalently bound to at least some sections of the pore boundary area of at least one of the pores.

18. The device according to claim 17, wherein the capture molecules are oligonucleotide probes which are bound via terminal amino or thiol groups to linker molecules which in turn are bound via covalent and/or ionic groups to the pore boundary area.

19. A use of a device according to claim 1 as a basis for a sample support in methods of detecting biochemical reactions and/or bindings and also for studying enzymic reactions, nucleic acid hybridizations, protein-protein interactions, and protein-ligand interactions.

20. A method of detecting chemical or biochemical reactions and/or bindings, comprising the steps of:
providing a device according to claim 17;
introducing a substance to be studied into at least one of the pores of the support material, with the refractive index nₚₒᵣₑ of the substance to be studied being less than the refractive index n_{waveguide} of the first layer;
coupling out luminescence light of the substance to be studied; and
studying the luminescence light of the material to be studied.

21. The method according to claim 20, further comprising the step of coupling excitation light as waveguide modes into the first layer of the layered structure in order to excite the material to be studied at the pore boundary area in an evanescent field of the waveguide modes.

22. A method of detecting chemical or biochemical reactions and/or bindings, comprising the steps of:
providing a device according to claim 18;
introducing a substance to be studied into at least one of the pores of the support material, with the refractive index nₚₒᵣₑ of the substance to be studied being less than the refractive index n_{waveguide} of the first layer;
coupling out luminescence light of the substance to be studied; and
studying the luminescence light of the material to be studied.

23. The method according to claim 22, further comprising the step of coupling excitation light as waveguide modes into the first layer of the layered structure in order to excite the material to be studied at the pore boundary area in an evanescent field of the waveguide modes.

24. A method of detecting chemical or biochemical reactions and/or bindings, comprising the steps of:
providing a device according to claim 17;
introducing a substance to be studied into at least one of the pores of the support material, with the refractive index nₚₒᵣₑ of the substance to be studied being less than the refractive index n_{waveguide} of the first layer;
coupling excitation light as waveguide modes into the first layer of the layered structure in order to excite the substance to be studied at the pore boundary area in an evanescent field of the waveguide modes; and
studying the excitation light coupled out of the first layer, after interaction with the substance to be studied.

25. A method of detecting chemical or biochemical reactions and/or bindings, comprising the steps of:
providing a device according to claim 18;
introducing a substance to be studied into at least one of the pores of the support material, with the refractive index nₚₒᵣₑ of the substance to be studied being less than the refractive index n_{waveguide} of the first layer;
coupling excitation light as waveguide modes into the first layer of the layered structure in order to excite the substance to be studied at the pore boundary area in an evanescent field of the waveguide modes; and
studying the excitation light coupled out of the first layer, after interaction with the substance to be studied.

26. A method of preparing a device according to claim 1, comprising the steps of:
(a) providing a two-dimensional support material made of silicon and having two surfaces facing each other;
(b) generating blind holes whose depth is less than the thickness of the support material by electrochemically etching one of the surfaces of the support material;
(c) removing the support material from the opposite surface at least to the bottom of the blind holes to obtain pores which stretch throughout from the one surface to the opposite surface of the support material;
(d) subjecting the support material obtained in step (c) to an oxidation so that the pore walls of the support material consist, at least in the region close to the pores, of SiO₂ to form the second layer; and
(e) coating at least one of the pore walls in order to form the first layer.

27. A method of preparing a device comprising the steps of:
(a) providing a two-dimensional support material made of silicon and having two surfaces facing each other;
(b) generating blind holes whose depth is less than the thickness of the support material by electrochemically etching one of the surfaces of the support material;
(c) removing the support material from the opposite surface at least to the bottom of the blind holes to obtain pores which stretch throughout from the one surface to the opposite surface of the support material;
(d) subjecting the support material obtained in step (c) to an oxidation so that pore walls of the support material consist, at least in regions close to the pores, of SiO₂ to form a second layer; and
(e) coating at least one of the pore walls in order to form a first layer.

28. A method of detecting chemical or biochemical reactions and/or bindings, comprising the steps of:
providing a device having a two-dimensionally formed support material which has, spread across at least one surface area, a plurality of pores which stretch throughout from one surface of the support material to the opposite surface, with the pores being bounded by a pore boundary area of pore walls formed in the support material along the longitudinal axes of the pores and capture molecules being immobilized thereto, where appropriate via linker molecules;
introducing a substance to be studied into at least one of the pores of the support material, with the refractive index nₚₒᵣₑ of the substance to be studied being greater than the refractive index n_{pore wall} of the pore wall in the region of the pore boundary area;
coupling out luminescence light of the substance to be studied as waveguide modes of the pore forming a waveguide; and
studying the luminescence light of the substance to be studied.

29. The method according to claim 28, further comprising the step of coupling excitation light as waveguide modes into at least one of the pores in order to excite the substance to be studied.

30. A method of controlling chemical or biochemical reactions or syntheses, comprising the steps of:
providing a device according to claim 17;
introducing a synthetic substance into at least one of the pores of the support material, with the refractive index nₚₒᵣₑ of the synthetic substance being less than the refractive index n_{waveguide} of the first layer; and
coupling light as waveguide modes into the first layer of the layered structure in order to optically excite at least the synthetic substance at the pore boundary area in an evanescent field of the waveguide modes.

31. A method of controlling chemical or biochemical reactions or syntheses, comprising the steps of:
providing a device according to claim 18;
introducing a synthetic substance into at least one of the pores of the support material, with the refractive index nₚₒᵣₑ of the synthetic substance being less than the refractive index n_{waveguide} of the first layer; and
coupling light as waveguide modes into the first layer of the layered structure in order to optically excite at least the synthetic substance at the pore boundary area in an evanescent field of the waveguide modes.
